(19) United States
(12) Patent Application Publication
(10) Pub. No.: US 2024/0389983 A1
(43) Pub. Date: Nov. 28, 2024

(54) WEARABLE FERTILITY PREDICTION DEVICE

(71) Applicant: Jessica Ann Wojcik, Golden Valley, MN (US)

(72) Inventor: Jessica Ann Wojcik, Golden Valley, MN (US)

(21) Appl. No.: 18/671,538

(22) Filed: May 22, 2024

Related U.S. Application Data
(60) Provisional application No. 63/503,566, filed on May 22, 2023.

(51) Int. Cl. A61B 10/00; A61B 5/145; A61B 8/00; A61B 8/08; A61B 8/12

(52) U.S. Cl. CPC A61B 10/0012; A61B 8/085; A61B 8/12; A61B 8/4416; A61B 8/5223; A61B 8/56; A61B 5/14503; A61B 5/14514; A61B 5/14546; A61B 2010/0019

(57) ABSTRACT

This disclosure describes a wearable medical device. The device includes an attachment component that at least temporarily secures the wearable medical device to skin of a user. The device further includes one or more sensors configured to capture data indicative of one or more fertility characteristics of the user.

### RELATED APPLICATIONS

This application claims the benefit of U.S. Provisional Application No. 63/503,566, filed May 22, 2023, the entire contents of which are incorporated herein by reference.

### TECHNICAL FIELD

The disclosure relates to wearable medical devices, such as for fertility predictions.

### BACKGROUND OF THE INVENTION

In a more modern world, people with uteruses are gravitating away from oral contraceptives and towards natural family planning (NFP) or the fertility awareness method (FAM) as a growing number of lawsuits are filed against numerous contraceptive devices and medications claiming mental altercations, blood clots, heart attacks, liver cancer, strokes and more. Additionally, infertility rates are on the rise as a growing number of couples trying to conceive (TTC) pour an extraordinary amount of resources into growing their family. The solutions that exist today are limited and typically include taking basal body temperatures (BBTs) orally or vaginally before getting out of bed in the morning (which can be messy and requires discipline), urinating on ovulation strips (which can be messy and time consuming), tracking cervical mucous (which can be qualitative, variable from person to person, and requires consistent documenting), tracking by first day of LMP (which can be not always reliable, cycles vary, require consistent and accurate documenting), and ultrasound monitoring in clinic (which is hindered by limited accessibility and being expensive and time consuming).

### SUMMARY OF THE INVENTION

In general, the disclosure is directed to a wearable medical device. The wearable medical device at least temporarily gets secured to the user, such as via an adhesive layer on the wearable medical device or a strap. The wearable medical device also includes one or more sensors, such as hormone-detecting sensors connected to microneedles, temperature sensors, and/or ultrasound cameras, configured to measure data indicative of one or more fertility characteristics, the data including any one or more of one or more hormone levels in the interstitial fluid beneath the skin of the user, basal body temperature (BBT), or ultrasound images of the user's ovaries and/or endometrium.

This technology provides a number of benefits to those with infertility, those who may be trying to conceive (TTC), or those who are actively trying not to conceive. Many current products to predict ovulation in a person having ovaries face a number of problems. Ovulation prediction kits sold over the counter require a user to urinate either onto a stick that is held by the user or into a vessel that a user must place on a counter and then dip the stick into the vessel. This can be unsanitary and messy, and the results are often difficult to ascertain without a reference to a guide. Using a thermometer to take a BBT is also unreliable, as BBT must be taken at specific times in relation to the user waking up and any activity or time difference between the user waking up and taking the BBT can lead to misleading results. This can be especially troublesome as BBT requires a true trend to be detected with comparisons to previous data entries, meaning accurate measurements are very important. With ultrasound machines, many typical users are not trained to operate these machines, read the results, or afford to purchase one for a home. While ultrasound machines may be reliable, large, medical grade ultrasound machines are not realistic options for the average consumer.

This technology solves many of the above issues. In instances where microneedles are implemented into the wearable medical device, the microneedles and hormone-detecting sensors may measure hormone levels in interstitial fluid of the user, removing the need to hold urine overnight or to handle one's own (or another individual's) urine to measure the hormone levels with a stick. In instances where temperature sensors are used, the wearable medical device can measure the temperature at a same absolute or relative time each day (e.g., a same time each morning, within a certain amount of minutes of a user waking up, or within a certain amount of minutes of an alarm set by the user), obtaining reliable results that can be accurately tracked each day. In instances where ultrasound sensors are incorporated into the wearable medical device, the wearable medical device may be at least temporarily attached to a person at a specific position that will accurately gather one or more images of key parts of a person with ovaries, including the ovaries themselves and the endometrium. Detecting dominant follicles, a thickened and trilaminar endometrium, and a corpus luteum cyst (CLC) within ultrasound images may be quite reliable, so having a model trained to detect these structures in an ultrasound image captured by a wearable medical device would lead to accurate and accessible fertility information.

In one example, the disclosure is directed to a wearable medical device comprising an attachment component that at least temporarily secures the wearable medical device to skin of a user and one or more sensors configured to capture data indicative of one or more fertility characteristics of the user.

In another example, the disclosure is directed to a method comprising securing a wearable medical device of to a user, the wearable medical device comprising an attachment component that at least temporarily secures the wearable medical device to skin of the user and one or more sensors configured to capture data indicative of one or more fertility characteristics of the user. The method further comprises receiving, by one or more processors, the data indicative of the one or more fertility characteristics of the user from the wearable medical device. The method also comprises applying, by the one or more processors, one or more artificial intelligence models to the data. The method further comprises determining, by the one or more processors, one or more fertility characteristics of the user.

In another example, the disclosure is directed to a non-transitory computer-readable storage medium comprising instructions that, when executed by one or more processors, cause the one or more processors to control one or more sensors of a wearable medical device secured to a user to capture data indicative of one or more fertility characteristics of the user. The instructions, when executed, further cause the one or more processors to log the data in a database as part of a series of respective data measurements. The instructions, when executed, also cause the one or more processors to apply one or more models to the series of respective data measurements to determine a trend for the series of respective data measurements. The instructions, when executed, further cause the one or more processors to, based on the trend for the series of respective data measurements, determine the one or more fertility characteristics of the user.

In another example, the disclosure is directed to a secondary computing device and the wearable medical device described above.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

The following drawings are illustrative of particular examples of the present disclosure and therefore do not limit the scope of the invention. The drawings are not necessarily to scale, though examples can include the scale illustrated, and are intended for use in conjunction with the explanations in the following detailed description wherein like reference characters denote like elements. Examples of the present disclosure will hereinafter be described in conjunction with the appended drawings.

FIG. 1 is a conceptual diagram illustrating a user wearing a wearable medical device in accordance with the disclosure.

FIG. 2 is a block diagram illustrating a more detailed example of a computing device configured to perform the techniques described herein.

FIGS. 3A-3C are conceptual diagrams illustrating different views of the wearable medical device described herein.

FIG. 4 is a flow diagram illustrating an example process for measuring one or more fertility characteristics of a patient, in accordance with one or more techniques of this disclosure.

FIG. 5 is a chart showing various fertility characteristic levels throughout a typical menstruation cycle, in accordance with one or more techniques of this disclosure.

### DETAILED DESCRIPTION

The following detailed description is exemplary in nature and is not intended to limit the scope, applicability, or configuration of the techniques or systems described herein in any way. Rather, the following description provides some practical illustrations for implementing examples of the techniques or systems described herein. Those skilled in the art will recognize that many of the noted examples have a variety of suitable alternatives.

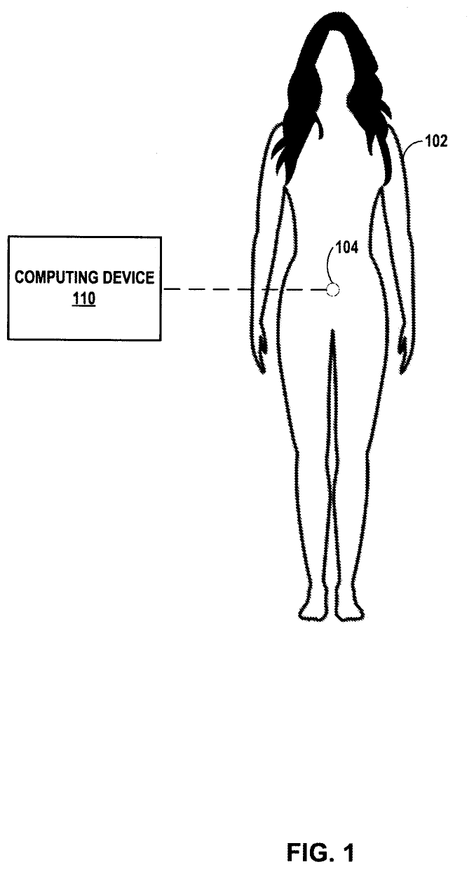

FIG. 1 is a conceptual diagram illustrating a user 102 wearing a wearable medical device 104 in accordance with the disclosure. Wearable medical device 104 may include an adhesive layer to adhere wearable medical device 104 to user 102. In other instances, wearable medical device may be attached by additional or alternative methods, including by being attached to a strap that wraps around the user.

In some instances, wearable medical device 104 further includes one or more microneedles that penetrate skin of user 102 such that the one or more microneedles are in contact with interstitial fluid of user 102.

Wearable medical device 104 may further include one or more sensors, including any one or more of hormone-detecting sensors (also referred to herein as hormone detection or hormone concentration sensors), temperature sensors, and/or ultrasound sensors, among other things. While wearable medical device 104 is located on the center of user 102's torso just above the pubic bone of user 102, wearable medical device 104 may be worn at other locations in other instances, such as an arm, a leg, off-center on user 102's torso, or a hip of user 102.

A hormone-detecting sensor may be any sensor capable of receiving fluid and determining an amount or concentration of a particular hormone in the fluid. A temperature sensor may be any sensor capable of detecting a temperature, such as a basal body temperature (BBT), of a human, including infrared temperature sensors or contact temperature sensors. An ultrasound sensor may be any sensor capable of producing an ultrasound image of internal tissue of a user, including portable and/or wearable ultrasound imaging cameras, devices, or patches.

Computing device 110 may be any computer with the processing power required to adequately execute the techniques described herein. For instance, computing device 110 may be any one or more of a mobile computing device (e.g., a smartphone, a tablet computer, a laptop computer, etc.), a desktop computer, an integrated computing device, a smarthome component (e.g., a computerized appliance, a home security system, a control panel for home components, a lighting system, a smart power outlet, etc.), a wearable computing device (e.g., a smart watch, computerized glasses, a heart monitor, a glucose monitor, smart headphones, etc.), a virtual reality/augmented reality/extended reality (VR/AR/XR) system, a video game or streaming system, a network modem, router, or server system, or any other computerized device that may be configured to perform the techniques described herein. In some instances, computing device 110 may be a separate device than wearable medical device 104. In other instances, all or part of computing device 110 may be physically incorporated into wearable medical device 104.

In accordance with the techniques and devices of this disclosure, an artificial intelligence (AI)-enabled, a wearable medical patch (i.e., wearable medical device 104) is described. In certain instances, the wearable patch may be capable of unifying the ‘trifecta’ of cycle tracking data: basal body temperature, blood hormone levels, and real-time ultrasound monitoring to provide an effortless and highly accurate projection of a person's fertile window.

The wearable AI-enabled patch may track a number of fertility characteristics of a user, including basal body temperature (BBT), blood hormone levels, maturation of dominant follicle on ovaries, endometrial thickness, and presence of free fluid in the pelvis through AI-driven ultrasound imaging to provide a judiciously accurate fertile window foresight.

When wearable medical device 104 is configured to measure BBT of the patient, wearable medical device 104 may be configured to measure the BBT minutes before movement is detected every morning, or on-demand.

Wearable medical device 104 may, additionally or alternatively, use microneedle technology to collect small samples of blood or interstitial fluid to test one or more of luteinizing hormone (LH) and follicle stimulating hormone (FSH) levels. A LH surge (usually of at least 25 mUI/ml) indicates ovulation will likely take place within 12-36 hours later. LH starts surging in the blood and interstitial fluid between 12 am and 8 am, and in the urine 3-6 hours later. For this reason, the blood/interstitial fluid sample may also be collected at the same time as BBT early in the morning before the consumer gets up to empty their bladder in instances where wearable medical device 104 is configured to measure both characteristics.

In some instances, wearable medical device 104 may, additionally or alternatively, include ultrasound technology to capture ultrasound images of user 102 at either on-demand or pre-defined times. In such instances, wearable medical device 104 may be placed midline, superior to the pubic bone. An ultrasound sweep through the uterus and ovaries every morning before the urinary bladder is emptied will provide AI-driven data about changes to the dominant follicle(s) on the ovaries, the thickness of the endometrium, the appearance of the endometrium (e.g., trilaminar vs. early proliferative), and any free fluid present adjacent to the ovaries and uterus. By taking the sweep before the urinary bladder is emptied, the full bladder will act as an acoustic window to better visualize the uterus and ovaries of the user.

In some instances, wearable medical device 104 may further send any recorded data to a physician of user 102. This may be helpful for consumers with infertility or who may be trying to conceive (TTC) and would like quantitative collected data sent to providers.

In addition to the functions described above, user 102 may further utilize one or more of wearable medical device 104 and computing device 110 to add a viewer (partner) to the account, send data to the physician, and collect data more frequently.

In some instances, computing device 110 may utilize a software application as an interface to view and analyze data recorded by wearable medical device 104. The software application may provide real-time updates to the individual and their partner and/or provider if desired, such as by communicating directly with computing devices associated with the partner and/or provider or by sending updates to an account on a cloud service associated with the individual, which then pushes the updates to computing devices associated with the partner and/or provider.

Instead of solely displaying quantitative data which may be overwhelming or difficult to interpret for consumer without background knowledge of BBT, blood hormone levels, and ultrasound findings, the software application may have two dashboards.

A first view may include a color coded chart or display with a marker that shows where the user falls in their cycle based on the compiled data. This could be in the form of a small “you are here” marker that shows the individual the precise location where they fall in their cycle. A second view may include a display that shows progression of data over time with every acquired temperature, lab value, ultrasound measurements, etc. Additionally or alternatively, the views may include a simple description of a predicted stage of ovulation the user is currently in, such as pre-ovulation, within a certain number of hours of potential ovulation, recently ovulated, luteal phase, or period.

The patch may be available in different colors that resemble various skin tones (similar to different shades of bandages) so that they are discreet and not noticeable when worn.

Using the ultrasound technology, wearable medical device 104 could also detect pelvic pathology such fibroids, polyps, or ovarian pathology as a result of the AI-ultrasound algorithms that are built into the patch. Each of these structures may have certain distinct shapes that, when detected by the various models and processors described herein, may indicate presence of such fibroids, polyps, or ovarian pathology.

An individual's BBT typically rises less than ½ degree Fahrenheit when ovulation takes place. For most the accurate readings, BBT should be measured at the same time each day, before getting out of bed, and after at least three consecutive hours of sleep.

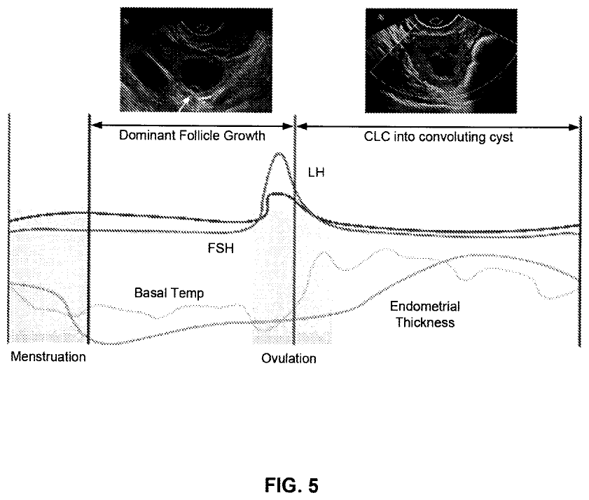

FIG. 5 is a chart showing various fertility characteristic levels throughout a typical menstruation cycle, in accordance with one or more techniques of this disclosure. There are four main hormones monitored to predict ovulation and fertility: Luteinizing Hormone (LH), Follicle Stimulating Hormone (FSH), Estrogen, and Progesterone (see below). These are most often tested using urine analysis strips but can also be identified in blood draws. Table 1, shown below, indicates how each of these hormones change throughout a person's typical cycle:

TABLE 1
Trends of levels for various hormones throughout menstrual cycle

| Hormone | Trend |
|---|---|
| Luteinizing Hormone (LH) | Surges pre-ovulation |
| Follicle Stimulating Hormone (FSH) | Surges pre-ovulation |
| Estrogen | Decreases post-ovulation |
| Progesterone | Increases post-ovulation |

The advantage of ultrasound is that it can monitor and ensure proper cycle progression from day one through the final day of a person's cycle. This is done through evaluation of ovarian follicles, endometrial thickness and appearance, and presence of free fluid in the adjacent adnexa and posterior cul-de-sac. Below are examples of some sonographic changes that can be seen as people with uteruses approach ovulation.

Typically, the ovaries alternate sides every month to grow a dominant follicle. Between 2-2.5 cm is typically when a dominant follicle ruptures and releases the ovum. Pre-ovulation, this dominant follicle can be seen growing daily by millimeters. During this time it has an anechoic, simple-appearing cyst (e.g., black on the ultrasound) appearance. As soon as the dominant follicle ruptures, the appearance of the ovary changes and instead of an anechoic follicle, a heterogeneous corpus luteum cyst can be visualized where the simple-appearing dominant follicle once was.

In some instances, computing device 110 and/or wearable medical device 104 may utilize Color Doppler or Color Flow (CF) assessment to identify features of a corpus luteum cyst (CLC). When a dominant follicle ruptures and leaves behind a corpus luteum cyst, CF may be used to confirm CLC presence. On ultrasound, this rupturing may appear as a hyper-vascular “ring”, sometimes known as the “ring of fire” around the periphery of the CLC. This blood flow is typically low resistance, which makes it easy to identify with proper Doppler imaging settings (baseline, color scale, etc.). A CF setting with parameters specifically designed for CLC detection will assist in confident confirmation of the CLC.

In determining the CLC or the dominant follicle, computing device 110 and/or wearable medical device 104 may utilize artificial intelligence systems to make ascertain the nature of the characteristics determined from the ultrasound. For instance, the system may take any one or more of the ultrasound, the basal body temperature, and hormone measurements from wearable medical device 104. Based on prior instances of the user ovulating or based on previous trainings of AI models based on ultrasound characteristic trends, basal body temperature trends, and hormone measurement trends, computing device 110 and/or wearable medical device 104 may determine whether the user is not close to ovulating, about to ovulate, currently ovulating, or past ovulation. In instances where ultrasounds are included in the input to these AI models, computing device 110 and/or wearable medical device 104 may identify the dominant follicle within the ultrasound and track its change in appearance from a simple-appearing cyst to a CLC with a heterogeneous appearance and a “ring of fire” around its periphery.

The endometrial thickness will gradually increase approaching ovulation. Once ovulation occurs the endometrial thickness will then increase more rapidly than prior to ovulation and will peak just before menstruation occurs. At the time of menstruation, the endometrial lining sheds and the cycle begins again.

Free fluid adjacent to the ovary or in the posterior cul-de-sac is often appreciated on ultrasound examination immediately after ovulation.

Wearable medical device 104 may prove to be beneficial for menstruating people wanting to prevent pregnancy (approx. ages 13-50), the trying to conceive (TTC) demographic, couples struggling with infertility, menstruating people interested in better understanding and tracking their cycle, and teens to middle aged people with uteruses wanting a safer alternative to oral contraceptives. This demographic also happens to be the category consuming the most content on social media on a daily basis. Wellness and the pursuit of “clean” living to support natural hormone regulation is undoubtedly trending.

Wearable medical device 104 may further be beneficial for partners of people with uteruses TTC. When a couple is trying to conceive, it is vital that both individuals have information regarding fertile windows and ovulation. With a synced app, partners of people with uteruses TTC will have access to ovulation data.

Wearable medical device 104 may further be beneficial for teens to middle aged people with uteruses interested in accurate tracking of their cycle for NFP, simply to estimate monthly cycle arrival, or irregularities that may point to gynecological pathology or disorders.

This technology provides a number of benefits to those with infertility, those who may be trying to conceive (TTC), or those who are actively trying not to conceive. Many current products to predict ovulation in a person having ovaries face a number of problems. Ovulation prediction kits sold over the counter require a user to urinate either onto a stick that is held by the user or into a vessel that a user must place on a counter and then dip the stick into the vessel. This can be unsanitary and messy, and the results are often difficult to ascertain without a reference to a guide. Using a thermometer to take a BBT is also unreliable, as BBT must be taken at specific times in relation to the user waking up and any activity or time difference between the user waking up and taking the BBT can lead to misleading results. This can be especially troublesome as BBT requires a true trend to be detected with comparisons to previous data entries, meaning accurate measurements are very important. With ultrasound machines, many typical users are not trained to operate these machines, read the results, or afford to purchase one for a home. While ultrasound machines may be reliable, large, medical grade ultrasound machines are not realistic options for the average consumer.

This technology solves many of the above issues. In instances where microneedles are implemented into the wearable medical device, the microneedles and hormone-detecting sensors may measure hormone levels in interstitial fluid of the user, removing the need to hold urine overnight or to handle one's own (or another individual's) urine to measure the hormone levels with a stick. In instances where temperature sensors are used, the wearable medical device can measure the temperature at a same absolute or relative time each day (e.g., a same time each morning, within a certain amount of minutes of a user waking up, or within a certain amount of minutes of an alarm set by the user), obtaining reliable results that can be accurately tracked each day. In instances where ultrasound sensors are incorporated into the wearable medical device, the wearable medical device may be at least temporarily attached to a person at a specific position that will accurately gather one or more images of key parts of a person with ovaries, including the ovaries themselves and the endometrium. Detecting dominant follicles, a thickened and trilaminar endometrium, and a corpus luteum cyst (CLC) within ultrasound images may be quite reliable, so having a model trained to detect these structures in an ultrasound image captured by a wearable medical device would lead to accurate and accessible fertility information.

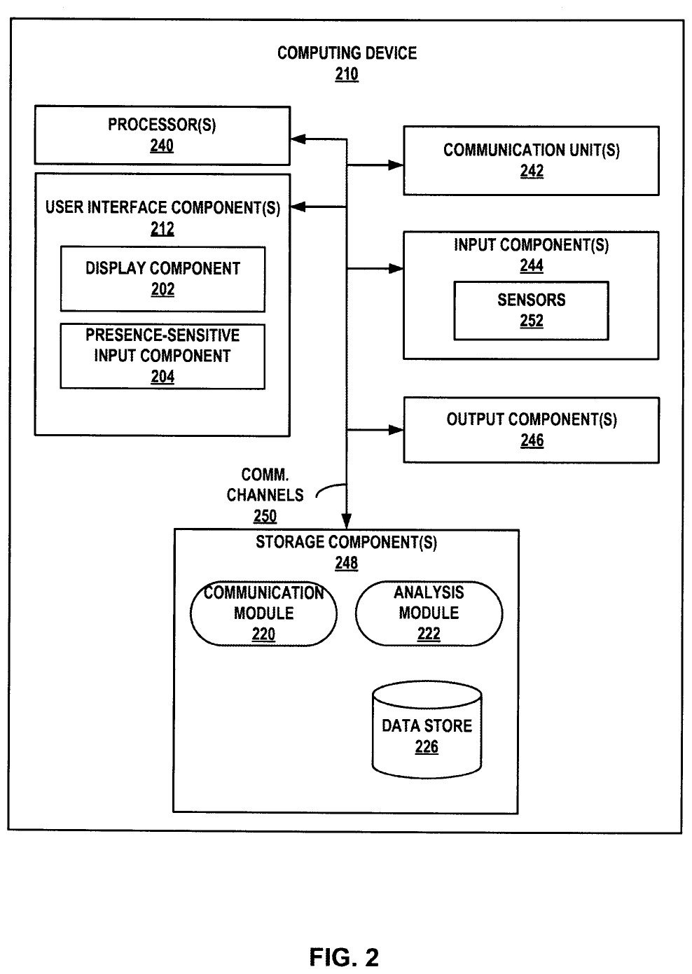

FIG. 2 is a block diagram illustrating a more detailed example of a computing device configured to perform the techniques described herein. Computing device 210 of FIG. 2 is described below as an example of computing device 110 of FIG. 1. FIG. 2 illustrates only one particular example of computing device 210, and many other examples of computing device 210 may be used in other instances and may include a subset of the components included in example computing device 210 or may include additional components not shown in FIG. 2.

Computing device 210 may be any computer with the processing power required to adequately execute the techniques described herein. For instance, computing device 210 may be any one or more of a mobile computing device (e.g., a smartphone, a tablet computer, a laptop computer, etc.), a desktop computer, a smarthome component (e.g., a computerized appliance, a home security system, a control panel for home components, a lighting system, a smart power outlet, etc.), a wearable computing device (e.g., a smart watch, computerized glasses, a heart monitor, a glucose monitor, smart headphones, etc.), a virtual reality/augmented reality/extended reality (VR/AR/XR) system, a video game or streaming system, a network modem, router, or server system, or any other computerized device that may be configured to perform the techniques described herein.

As shown in the example of FIG. 2, computing device 210 includes user interface components (UIC) 212, one or more processors 240, one or more communication units 242, one or more input components 244, one or more output components 246, and one or more storage components 248. UIC 212 includes display component 202 and presence-sensitive input component 204. Storage components 248 of computing device 210 include communication module 220, analysis module 222, and data store 226.

One or more processors 240 may implement functionality and/or execute instructions associated with computing device 210 to communicate with sensors and evaluate various characteristics of a user's body to determine a fertile window for the user. That is, processors 240 may implement functionality and/or execute instructions associated with computing device 210 to communicate with sensors and evaluate various characteristics of a user's body to determine a fertile window for the user.

Examples of processors 240 include any combination of application processors, display controllers, auxiliary processors, one or more sensor hubs, and any other hardware configured to function as a processor, a processing unit, or a processing device, including dedicated graphical processing units (GPUs). Modules 220 and 222 may be operable by processors 240 to perform various actions, operations, or functions of computing device 210. For example, processors 240 of computing device 210 may retrieve and execute instructions stored by storage components 248 that cause processors 240 to perform the operations described with respect to modules 220 and 222. The instructions, when executed by processors 240, may cause computing device 210 to communicate with sensors and evaluate various characteristics of a user's body to determine fertility characteristics for the user.

Communication module 220 may execute locally (e.g., at processors 240) to provide functions associated with retrieving user data from sensors and outputting notifications. In some examples, communication module 220 may act as an interface to a remote service accessible to computing device 210. For example, communication module 220 may be an interface or application programming interface (API) to a remote server that communicates with sensors and output devices.

In some examples, analysis module 222 may execute locally (e.g., at processors 240) to analyze the data received by communication module to determine a fertile window of a user, among other things. In some examples, analysis module 222 may act as an interface to a remote service accessible to computing device 210. For example, analysis module 222 may be an interface or application programming interface (API) to a remote server that analyzes data received from various sensors of a wearable medical device to determine fertility characteristics of the user.

One or more storage components 248 within computing device 210 may store information for processing during operation of computing device 210 (e.g., computing device 210 may store data accessed by modules 220 and 222 during execution at computing device 210). In some examples, storage component 248 is a temporary memory, meaning that a primary purpose of storage component 248 is not long-term storage. Storage components 248 on computing device 210 may be configured for short-term storage of information as volatile memory and therefore not retain stored contents if powered off. Examples of volatile memories include random access memories (RAM), dynamic random access memories (DRAM), static random access memories (SRAM), and other forms of volatile memories known in the art.

Storage components 248, in some examples, also include one or more computer-readable storage media. Storage components 248 in some examples include one or more non-transitory computer-readable storage mediums. Storage components 248 may be configured to store larger amounts of information than typically stored by volatile memory. Storage components 248 may further be configured for long-term storage of information as non-volatile memory space and retain information after power on/off cycles. Examples of non-volatile memories include magnetic hard discs, optical discs, floppy discs, flash memories, or forms of electrically programmable memories (EPROM) or electrically erasable and programmable (EEPROM) memories. Storage components 248 may store program instructions and/or information (e.g., data) associated with modules 220 and 222 and data store 226. Storage components 248 may include a memory configured to store data or other information associated with modules 220 and 222 data store 226.

Communication channels 250 may interconnect each of the components 212, 240, 242, 244, 246, and 248 for inter-component communications (physically, communicatively, and/or operatively). In some examples, communication channels 250 may include a system bus, a network connection, an inter-process communication data structure, or any other method for communicating data.

One or more communication units 242 of computing device 210 may communicate with external devices via one or more wired and/or wireless networks by transmitting and/or receiving network signals on one or more networks. Examples of communication units 242 include a network interface card (e.g., such as an Ethernet card), an optical transceiver, a radio frequency transceiver, a GPS receiver, a radio-frequency identification (RFID) transceiver, a near-field communication (NFC) transceiver, or any other type of device that can send and/or receive information. Other examples of communication units 242 may include short wave radios, cellular data radios, wireless network radios, as well as universal serial bus (USB) controllers.

One or more input components 244 of computing device 210 may receive input. Examples of input are tactile, audio, and video input. Input components 244 of computing device 210, in one example, include a presence-sensitive input device (e.g., a touch sensitive screen, a PSD), mouse, keyboard, voice responsive system, camera, microphone or any other type of device for detecting input from a human or machine. In some examples, input components 244 may include one or more sensor components (e.g., sensors 252). Sensors 252 may include one or more biometric sensors (e.g., fingerprint sensors, retina scanners, vocal input sensors/microphones, facial recognition sensors, cameras), one or more location sensors (e.g., GPS components, Wi-Fi components, cellular components), one or more temperature sensors, one or more movement sensors (e.g., accelerometers, gyros), one or more pressure sensors (e.g., barometer), one or more ambient light sensors, and one or more other sensors (e.g., infrared proximity sensor, hygrometer sensor, and the like). Other sensors, to name a few other non-limiting examples, may include a radar sensor, a lidar sensor, a sonar sensor, a heart rate sensor, magnetometer, glucose sensor, olfactory sensor, compass sensor, or a step counter sensor.

One or more output components 246 of computing device 210 may generate output in a selected modality. Examples of modalities may include a tactile notification, audible notification, visual notification, machine generated voice notification, or other modalities. Output components 246 of computing device 210, in one example, include a presence-sensitive display, a sound card, a video graphics adapter card, a speaker, a cathode ray tube (CRT) monitor, a liquid crystal display (LCD), a light emitting diode (LED) display, an organic LED (OLED) display, a virtual/augmented/extended reality (VR/AR/XR) system, a three-dimensional display, or any other type of device for generating output to a human or machine in a selected modality.

UIC 212 of computing device 210 may include display component 202 and presence-sensitive input component 204. Display component 202 may be a screen, such as any of the displays or systems described with respect to output components 246, at which information (e.g., a visual indication) is displayed by UIC 212 while presence-sensitive input component 204 may detect an object at and/or near display component 202.

While illustrated as an internal component of computing device 210, UIC 212 may also represent an external component that shares a data path with computing device 210 for transmitting and/or receiving input and output. For instance, in one example, UIC 212 represents a built-in component of computing device 210 located within and physically connected to the external packaging of computing device 210 (e.g., a screen on a mobile phone). In another example, UIC 212 represents an external component of computing device 210 located outside and physically separated from the packaging or housing of computing device 210 (e.g., a monitor, a projector, etc. that shares a wired and/or wireless data path with computing device 210).

UIC 212 of computing device 210 may detect two-dimensional and/or three-dimensional gestures as input from a user of computing device 210. For instance, a sensor of UIC 212 may detect a user's movement (e.g., moving a hand, an arm, a pen, a stylus, a tactile object, etc.) within a threshold distance of the sensor of UIC 212. UIC 212 may determine a two or three-dimensional vector representation of the movement and correlate the vector representation to a gesture input (e.g., a hand-wave, a pinch, a clap, a pen stroke, etc.) that has multiple dimensions. In other words, UIC 212 can detect a multi-dimension gesture without requiring the user to gesture at or near a screen or surface at which UIC 212 outputs information for display. Instead, UIC 212 can detect a multi-dimensional gesture performed at or near a sensor which may or may not be located near the screen or surface at which UIC 212 outputs information for display.

In accordance with the techniques of this disclosure, computing device 210 may perform any of the methods or techniques described herein, including controlling any sensors, analyzing any data, and outputting any notifications or data analyses as described with respect to the wearable medical device described throughout this disclosure and the claims.

Computing device 210 may be incorporated or integrated into a wearable medical device, such as wearable medical device 104 of FIG. 1. In other instances, computing device 210 may be a secondary computing device, such as a smartphone, a tablet computer, or a server, in communication with the wearable medical device. The wearable medical device may include an attachment component that at least temporarily secures the wearable medical device to skin of a user and one or more sensors configured to capture data indicative of one or more fertility characteristics of the user.

In some instances, the one or more sensors (e.g., sensors 252 when computing device 210 is an integrated computing device) may include one or more hormone-detecting sensors configured to capture one or more hormone levels, either incorporated into or in fluid communication with one or more microneedles that extend out of the wearable medical device, wherein, when the attachment component is secured to the skin of the user, the one or more microneedles are inserted through the skin of the user such that the one or more microneedles contact interstitial fluid beneath the skin of the user from which the one or more hormone-detecting sensors measure the one or more hormone levels. Additionally or alternatively, the one or more sensors may also include one or more temperature sensors configured to capture a basal body temperature (BBT) of the user. Additionally or alternatively, the one or more sensors may include one or more ultrasound devices configured to capture one or more ultrasound images of the user.

Communication module 220 may control the one or more sensors to capture the data indicative of the one or more fertility characteristics. In doing so, communication module 220 may control the sensors to capture this data at any of a particular set of one or more times each day, in response to receiving an indication of user input at the wearable medical device, and in response to receiving an indication of user input at a secondary computing device.

The particular set of one or more times each day may be either absolute or relative. For instance, communication module 220 may control the one or more sensors to capture the data at a set, absolute time each day (e.g., 5 AM, 6 AM, 7 AM, or any other time suitable for obtaining the desired measurements). In other instances, the time may be relative. For example, the wearable medical device may include one or more accelerometers and/or gyrometers to detect motion of an individual while they are sleeping. If the wearable medical device determines that the user is about to wake up or has woken up, communication module 220 may control the one or more sensors to capture the data at that time. Similarly, if the user has one or more alarm notifications set on computing device 210, though those alarms may fluctuate day-to-day, communication module 220 may control the one or more sensors to capture the data at a time relative to the alarms (e.g., within 5, 10, 15, 20, or 30 minutes of the alarm, among other possible relative times).

When the one or more fertility characteristics include the one or more hormone levels, those hormone levels may include any one or more of a luteinizing hormone level, a follicle stimulating hormone, estrogen, progesterone, and a human chorionic gonadotropin level.

When the data includes the one or more ultrasound images include representations, the one or more fertility characteristics (e.g., the structures detected within the ultrasound images) may include one or more of a dominant follicle on an ovary, a corpus luteum cyst, an endometrium, and free fluid adjacent to the ovary or in a posterior cul-de-sac.

The one or more fertility characteristics, in general, may include any one or more of a fertile window of the user, an ovulation for the user, a pregnancy of the user, and a non-pregnancy of the user. In other words, computing device 210 and the wearable computing device may work to ascertain any one or more of these fertility characteristics through its continued use.

In some instances, the wearable medical device has a color chosen from one of a plurality of different skin tone shades.

In some instances, the attachment component may include a strap to wrap around a body of the user and/or an adhesive layer that at least temporarily adheres the wearable medical device to the skin of the user.

Once the data is captured by the wearable medical device, communication module 220 may receive the data via communication unit 242, either through an integrated circuit, some other wired connection, or a wireless connection (e.g., short-wave radio connections, Wi-Fi connections, ZigBee connections, Matter connections, or any other type of wireless connection). Communication module 220 may log the data in a database, such as data store 226 (either locally or via a cloud service) as part of a series of respective data measurements.

Analysis module 222 may apply one or more models to the series of respective data measurements to determine a trend for the series of respective data measurements. In some instances, each of the one or more models are applicable to a respective type of the data logged in the database, such as having one set of models for each of one or more hormone levels from interstitial fluid beneath the skin of the user, one set of models for a basal body temperature (BBT) of the user, and one set of models for an ultrasound image of the user. Any combination of these models may be utilized depending on the combination of sensors in the wearable medical device (e.g., just hormone detection models, just BBT models, just ultrasound models, hormone detection models and BBT models, hormone detection models and ultrasound models, BBT models and ultrasound models, or all of hormone detection models, BBT models, and ultrasound models). In some instances, analysis module 222 may update any of the one or more models based on previous trends for the respective type of the data.

Based on the trend for the series of respective data measurements, analysis module 222 may determine the one or more fertility characteristics of the user. In some instances, communication module 220 may output a notification to one or more of an alert device in the wearable medical device or a secondary computing device, the notification indicating the one or more fertility characteristics of the user.

In some instances, communication module 220 may transmit, via communication unit 242, the data to a secondary computing device for analysis.

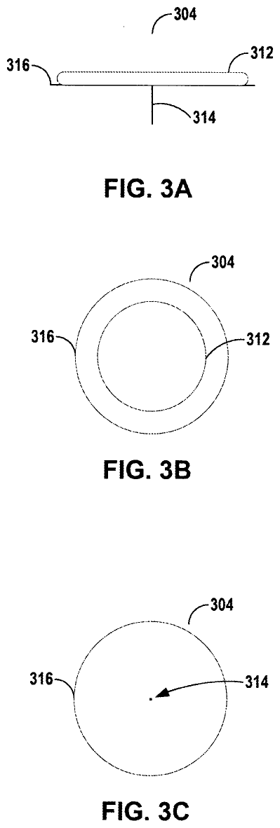

FIGS. 3A-3C are conceptual diagrams illustrating different views of the wearable medical device described herein. FIG. 3A is a side view of wearable medical device 304 showing electronics 312, microneedle 314, and adhesive layer 316. Adhesive layer 316 may include an adhesive on the underside of wearable medical device 304 such that wearable medical device can better adhere to skin of a user when the microneedle is inserted into the skin of the user. Any one or more of the sensors described herein may be incorporated into either microneedle 314 itself or electronics 312. Electronics 312 may include components such that electronics 312 are capable of being a computing device, such as computing device 210, or may include communication units such that wearable medical device 304 can communicate with a physically separate computing device and processors to process the data read by the various sensors. FIG. 3B is a top view of wearable medical device 304. FIG. 3C is a bottom view of wearable medical device 304.

While the example of FIGS. 3A-3C depict an example of wearable medical device 304 as having a single microneedle 314, it should be recognized that other configurations of wearable medical device 304 may exist. In some instances, wearable medical device 304 may not include a microneedle, instead relying on temperature and ultrasound measuring techniques alone separate from the hormone measurement techniques with microneedle 314. In other instances, wearable medical device 304 may include multiple microneedles, either of the same or of varying lengths, such as in instances where each microneedle is configured to measure a different hormone level for analysis by one or more processors as described herein.

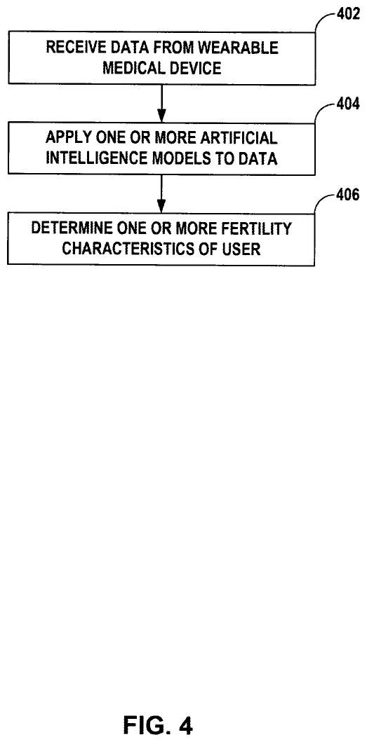

FIG. 4 is a flow diagram illustrating an example process for measuring one or more fertility characteristics of a patient, in accordance with one or more techniques of this disclosure. The techniques of FIG. 4 may be performed by one or more processors of a computing device, such as system 100 of FIG. 1 and/or computing device 210 illustrated in FIG. 2. For purposes of illustration only, the techniques of FIG. 4 are described within the context of computing device 210 of FIG. 4, although computing devices having configurations different than that of computing device 210 may perform the techniques of FIG. 4.

In accordance with the techniques of this disclosure, communication module 220 may receive data from a wearable medical device (402), such as wearable medical device 104 of FIG. 1 or wearable medical device 304 of FIG. 3. Computing device 210 may either be a standalone device communicating wirelessly with the wearable medical device via communication module 220, or may be incorporated into the wearable medical device. Analysis module 222 may apply one or more artificial intelligence models to the data (404). Analysis module 222 may determine one or more fertility characteristics of the user (406).

Example 1. A wearable medical device comprising an attachment component that at least temporarily secures the wearable medical device to skin of a user; and one or more sensors configured to capture data indicative of one or more fertility characteristics of the user.

Example 2. The wearable medical device of example 1, wherein the one or more sensors comprise one or more of one or more hormone-detecting sensors configured to capture one or more hormone levels, either incorporated into or in fluid communication with one or more microneedles that extend out of the wearable medical device, wherein, when the attachment component is secured to the skin of the user, the one or more microneedles are inserted through the skin of the user such that the one or more microneedles contact interstitial fluid beneath the skin of the user from which the one or more hormone-detecting sensors measure the one or more hormone levels; one or more temperature sensors configured to capture a basal body temperature (BBT) of the user; and one or more ultrasound devices configured to capture one or more ultrasound images of the user.

Example 3. The wearable medical device of example 2, wherein the one or more hormone levels comprise one or more of: a luteinizing hormone level; a follicle stimulating hormone; estrogen; progesterone; and a human chorionic gonadotropin level.

Example 4. The wearable medical device of any one or more of examples 2-3, wherein the one or more ultrasound images include representations of one or more of a dominant follicle on an ovary; a corpus luteum cyst; an endometrium; and free fluid adjacent to the ovary or in a posterior cul-de-sac.

Example 5. The wearable medical device of any one or more of examples 1-4, further comprising one or more processors, wherein the one or more processors are configured to: log the data in a database as part of a series of respective data measurements; apply one or more models to the series of respective data measurements to determine a trend for the series of respective data measurements; and based on the trend for the series of respective data measurements, determine the one or more fertility characteristics of the user.

Example 6. The wearable medical device of example 5, wherein the one or more processors are further configured to: output a notification to one or more of an alert device in the wearable medical device or a secondary computing device, the notification indicating the one or more fertility characteristics of the user.

Example 7. The wearable medical device of any one or more of examples 5-6, wherein the each of the one or more models are applicable to a respective type of the data logged in the database, wherein the type of the data comprises one or more of: one or more hormone levels from interstitial fluid beneath the skin of the user, a basal body temperature (BBT) of the user, and an ultrasound image of the user.

Example 8. The wearable medical device of example 7, wherein the one or more processors are further configured to update any of the one or more models based on previous trends for the respective type of the data.

Example 9. The wearable medical device of any one or more of examples 5-8, wherein the one or more processors are configured to: transmit, via a communication unit, the data to a secondary computing device for analysis.

Example 10. The wearable medical device of any one or more of examples 5-9, wherein the one or more processors are configured to control the one or more sensors to capture the data indicative of the one or more fertility characteristics.

Example 11. The wearable medical device of example 10, wherein the processors are configured to control the one or more sensors to the data indicative of the one or more fertility characteristics in one or more of: at a particular set of one or more times each day, in response to receiving an indication of user input at the wearable medical device, and in response to receiving an indication of user input at a secondary computing device.

Example 12. The wearable medical device of any one or more of examples 1-11, wherein the one or more fertility characteristics comprise one or more of a fertile window of the user; an ovulation for the user; a pregnancy of the user; and a non-pregnancy of the user.

Example 13. The wearable medical device of any one or more of examples 1-12, wherein the wearable medical device has a color chosen from one of a plurality of different skin tone shades.

Example 14. The wearable medical device of any one or more of examples 1-13, wherein the attachment component comprises one or more of a strap to wrap around a body of the user, and an adhesive layer that at least temporarily adheres the wearable medical device to the skin of the user.

Example 15. A method comprising securing a wearable medical device of to a user, the wearable medical device comprising an attachment component that at least temporarily secures the wearable medical device to skin of the user; and one or more sensors configured to capture data indicative of one or more fertility characteristics of the user; receiving, by one or more processors, the data indicative of the one or more fertility characteristics of the user from the wearable medical device; applying, by the one or more processors, one or more artificial intelligence models to the data; and determining, by the one or more processors, one or more fertility characteristics of the user.

Example 16. A non-transitory computer-readable storage medium comprising instructions that, when executed by one or more processors, cause the one or more processors to control one or more sensors of a wearable medical device secured to a user to capture data indicative of one or more fertility characteristics of the user; log the data in a database as part of a series of respective data measurements; apply one or more models to the series of respective data measurements to determine a trend for the series of respective data measurements; and based on the trend for the series of respective data measurements, determine the one or more fertility characteristics of the user.

Example 17. The non-transitory computer-readable storage medium of example 16, wherein the instructions, when executed, further cause the one or more processors to output a notification to one or more of an alert device in the wearable medical device or a secondary computing device, the notification indicating the one or more fertility characteristics of the user.

Example 18. The non-transitory computer-readable storage medium of any one or more of examples 16-17, wherein the each of the one or more models are applicable to a respective type of the data logged in the database, wherein the type of the data comprises one or more of one or more hormone levels from interstitial fluid beneath the skin of the user, a basal body temperature (BBT) of the user, and an ultrasound image of the user.

Example 19. The non-transitory computer-readable storage medium of example 18, wherein the instructions, when executed, further cause the one or more processors to update any of the one or more models based on previous trends for the respective type of the data.

Example 20. The non-transitory computer-readable storage medium of any one or more of examples 16-19, wherein the instructions, when executed, cause the one or more processors to control the one or more sensors to the data indicative of the one or more fertility characteristics in one or more of at a particular set of one or more times each day, in response to receiving an indication of user input at the wearable medical device, and in response to receiving an indication of user input at a secondary computing device.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

It is contemplated that the various aspects, features, processes, and operations from the various embodiments may be used in any of the other embodiments unless expressly stated to the contrary. Certain operations illustrated may be implemented by a computer executing a computer program product on a non-transient, computer-readable storage medium, where the computer program product includes instructions causing the computer to execute one or more of the operations, or to issue commands to other devices to execute one or more operations.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structures or any other structure suitable for implementation of the techniques described herein. In addition, in some aspects, the functionality described herein may be provided within dedicated hardware and/or software modules configured for encoding and decoding, or incorporated in a combined codec. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a codec hardware unit or provided by a collection of interoperative hardware units, including one or more processors as described above, in conjunction with suitable software and/or firmware.

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as a pre-configured, stand-alone hardware element and/or as preprogrammed hardware elements (e.g., application specific integrated circuits, FPGAs, and digital signal processors), or other related components.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount-including any integer-between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

Various examples of the disclosure have been described. Any combination of the described systems, operations, or functions is contemplated. These and other examples are within the scope of the following claims.

1. A wearable medical device comprising:
an attachment component that at least temporarily secures the wearable medical device to skin of a user; and
one or more sensors configured to capture data indicative of one or more fertility characteristics of the user.

2. The wearable medical device of claim 1, wherein the one or more sensors comprise one or more of:
one or more hormone-detecting sensors configured to capture one or more hormone levels, either incorporated into or in fluid communication with one or more microneedles that extend out of the wearable medical device, wherein, when the attachment component is secured to the skin of the user, the one or more microneedles are inserted through the skin of the user such that the one or more microneedles contact interstitial fluid beneath the skin of the user from which the one or more hormone-detecting sensors measure the one or more hormone levels;
one or more temperature sensors configured to capture a basal body temperature (BBT) of the user; and
one or more ultrasound devices configured to capture one or more ultrasound images of the user.

3. The wearable medical device of claim 2, wherein the one or more hormone levels comprise one or more of:
a luteinizing hormone level;
a follicle stimulating hormone;
estrogen;
progesterone; and
a human chorionic gonadotropin level.

4. The wearable medical device of claim 2, wherein the one or more ultrasound images include representations of one or more of:
a dominant follicle on an ovary;
a corpus luteum cyst;
an endometrium; and
free fluid adjacent to the ovary or in a posterior cul-de-sac.

5. The wearable medical device of claim 1, further comprising one or more processors, wherein the one or more processors are configured to:
log the data in a database as part of a series of respective data measurements;
apply one or more models to the series of respective data measurements to determine a trend for the series of respective data measurements; and
based on the trend for the series of respective data measurements, determine the one or more fertility characteristics of the user.

6. The wearable medical device of claim 5, wherein the one or more processors are further configured to:
output a notification to one or more of an alert device in the wearable medical device or a secondary computing device, the notification indicating the one or more fertility characteristics of the user.

7. The wearable medical device of claim 5, wherein the each of the one or more models are applicable to a respective type of the data logged in the database, wherein the type of the data comprises one or more of:
one or more hormone levels from interstitial fluid beneath the skin of the user,
a basal body temperature (BBT) of the user, and
an ultrasound image of the user.

8. The wearable medical device of claim 7, wherein the one or more processors are further configured to update any of the one or more models based on previous trends for the respective type of the data.

9. The wearable medical device of claim 5, wherein the one or more processors are configured to:
transmit, via a communication unit, the data to a secondary computing device for analysis.

10. The wearable medical device of claim 5, wherein the one or more processors are configured to control the one or more sensors to capture the data indicative of the one or more fertility characteristics.

11. The wearable medical device of claim 10, wherein the processors are configured to control the one or more sensors to the data indicative of the one or more fertility characteristics in one or more of:
at a particular set of one or more times each day,
in response to receiving an indication of user input at the wearable medical device, and
in response to receiving an indication of user input at a secondary computing device.

12. The wearable medical device of claim 1, wherein the one or more fertility characteristics comprise one or more of:
a fertile window of the user;
an ovulation for the user;
a pregnancy of the user; and
a non-pregnancy of the user.

13. The wearable medical device of claim 1, wherein the wearable medical device has a color chosen from one of a plurality of different skin tone shades.

14. The wearable medical device of claim 1, wherein the attachment component comprises one or more of:
a strap to wrap around a body of the user, and
an adhesive layer that at least temporarily adheres the wearable medical device to the skin of the user.

15. A method comprising:
securing a wearable medical device of to a user, the wearable medical device comprising:
an attachment component that at least temporarily secures the wearable medical device to skin of the user; and
one or more sensors configured to capture data indicative of one or more fertility characteristics of the user;
receiving, by one or more processors, the data indicative of the one or more fertility characteristics of the user from the wearable medical device;
applying, by the one or more processors, one or more artificial intelligence models to the data; and
determining, by the one or more processors, one or more fertility characteristics of the user.

16. A non-transitory computer-readable storage medium comprising instructions that, when executed by one or more processors, cause the one or more processors to:
control one or more sensors of a wearable medical device secured to a user to capture data indicative of one or more fertility characteristics of the user;
log the data in a database as part of a series of respective data measurements;
apply one or more models to the series of respective data measurements to determine a trend for the series of respective data measurements; and
based on the trend for the series of respective data measurements, determine the one or more fertility characteristics of the user.

17. The non-transitory computer-readable storage medium of claim 16, wherein the instructions, when executed, further cause the one or more processors to:
output a notification to one or more of an alert device in the wearable medical device or a secondary computing device, the notification indicating the one or more fertility characteristics of the user.

18. The non-transitory computer-readable storage medium of claim 16, wherein the each of the one or more models are applicable to a respective type of the data logged in the database, wherein the type of the data comprises one or more of:
one or more hormone levels from interstitial fluid beneath the skin of the user,
a basal body temperature (BBT) of the user, and
an ultrasound image of the user.

19. The non-transitory computer-readable storage medium of claim 18, wherein the instructions, when executed, further cause the one or more processors to:
update any of the one or more models based on previous trends for the respective type of the data.

20. The non-transitory computer-readable storage medium of claim 16, wherein the instructions, when executed, cause the one or more processors to control the one or more sensors to the data indicative of the one or more fertility characteristics in one or more of:
at a particular set of one or more times each day,
in response to receiving an indication of user input at the wearable medical device, and
in response to receiving an indication of user input at a secondary computing device.
